(19) United States
(12) Patent Application Publication
(10) Pub. No.: US 2021/0236404 A1
(43) Pub. Date: Aug. 5, 2021

(54) COMPOSITIONS AND METHODS FOR HAIR

(71) Applicant: L'OREAL, Paris (FR)

(72) Inventors: Rachel FEREBEE, Morristown, NJ (US); Guojin ZHANG, Westfield, NJ (US); Ronak RUGHANI, Edison, NJ (US)

(21) Appl. No.: 17/151,558

(22) Filed: Jan. 18, 2021

Related U.S. Application Data
(60) Provisional application No. 62/968,788, filed on Jan. 31, 2020.

(30) Foreign Application Priority Data
Mar. 5, 2020 (FR) 2002221

(51) Int. Cl. A61K 8/60; A61K 8/24; A61K 8/55; A61Q 5/00

(52) U.S. Cl. CPC A61K 8/60; A61K 8/24; A61K 8/55; A61K 2800/10; A61K 2800/88; A61K 2800/805; A61Q 5/002

(57) ABSTRACT

The disclosure relates to compositions and methods for treating hair, protecting hair from damage, and/or for reducing hair damage. The compositions comprise at least one compound having at least one phosphate group and at least one sugar. The methods comprise applying the compositions to hair.

### TECHNICAL FIELD

The present disclosure relates to compositions and methods for treating hair, protecting hair, or reducing damage to hair.

### BACKGROUND

Individuals desire healthy and strong hair, as healthy-looking hair is in general considered to be a sign of good health and good hair-care practices. However, nutrition, environmental influences, heat, and chemical hair treatments can lead to hair damage that significantly weakens and dulls the hair over time. In particular, conventional styling processes may be damaging to hair, such as heat-styling hair with heat tools (e.g. flat iron, curling iron) or chemical styling treatments (e.g. perming, relaxing). Hair that has been weakened or damaged is also prone to be frizzy, to have breakage, and to the formation of “split ends.”

Thus, there is a need for compositions and methods for protecting hair, wherein the compositions have desired cosmetic benefits and composition properties that protect hair from damage or reduce hair damage, such as heat-induced damage.

### SUMMARY

It has surprisingly been found that compositions according to the disclosure protect hair from damage or reduce hair damage from heat treatments, such as heat treatments with conventional heat tools (e.g. flat iron, curling iron).

The compositions may comprise at least one compound having at least one phosphate group and at least one sugar.

In an embodiment, the at least one sugar may be present in an amount of up to 6%, such as less than 6%, relative to the total weight of the composition.

In an embodiment, the at least one compound having at least one phosphate group may be present in an amount up to about 4.5%, or ranging from about 1% to about 0.2%, or ranging from about 0.01% to about 0.2%, relative to the total weight of the composition.

The at least one compound having at least one phosphate group may be chosen from monosodium phosphate, phytic acid or glycerol phosphate, or combinations thereof. In a further embodiment, the at least one compound having at least one phosphate group is phytic acid. In a further embodiment, the at least one compound having at least one phosphate group is glycerol phosphate. In a further embodiment, the at least one compound having at least one phosphate group is monosodium phosphate. When the at least one phosphate group is phytic acid, the phytic acid may be present in an amount ranging from about 0.001% to about 10%, relative to the total weight of the composition. When the at least one phosphate group is glycerol phosphate, the glycerol phosphate may be present in an amount ranging from about 0.001% to about 10%, relative to the total weight of the composition. When the at least one phosphate group is monosodium phosphate, the monosodium phosphate may be present in an amount ranging from about 0.001% to about 2%, relative to the total weight of the composition.

The at least one sugar may be chosen from C3-C6 monosaccharides. The at least one sugar may be chosen from xylose, arabinose, ribose, 2-deoxy-ribose, or combinations thereof. The at least one sugar may be chosen from allose, altrose, glucose, glucosamine, mannose, gulose, idose, galactose, talose, sorbose, psicose, fructose, tagatose, or combinations thereof. The at least one sugar may be chosen from xylose, glucose, or combinations thereof.

In an embodiment, the weight ratio of the total amount of the at least one phosphate to the total amount of the at least one sugar ranges from about 1:30 to about 30:1, from about 1:25 to about 25:1, from about 1:15 to about 15:1, from about 1:10 to about 10:1, from about 1:5 to about 5:1, from about 1:2 to about 2:1.

In a further embodiment, the composition comprises from about 50% to about 99% solvent, or from about 50% to about 99% water, by weight relative to the total composition.

In an embodiment, the pH of the composition is less than or equal to about 7.5.

In a further embodiment the hair protecting composition comprises: a) at least C3-C6 monosaccharide, wherein the at least one C3-C6 monosaccharide is present in an amount up to about 5% by weight, relative to the total weight of the composition, and b) at least one compound having at least one phosphate group chosen from: a. phytic acid, wherein the phytic acid is present in an amount up to about 4.5%, relative to the total weight of the composition, b. glycerol phosphate, wherein the glycerol phosphate is present in an amount ranging up to about 4.5%, relative to the total weight of the composition, or c. monosodium phosphate, wherein the monosodium phosphate is present in an amount ranging from about 1% to about 0.2%, relative to the total weight of the composition.

Methods of treating the hair or protecting the hair from heat-induced damage or reducing heat-induced damage to hair may comprise applying a composition comprising at least one sugar and at least one compound having at least one phosphate group onto hair.

Methods of treating the hair or protecting the hair from heat-induced damage or reducing heat-induced damage to hair may comprise (1) applying to the hair a hair protecting composition comprising at least one sugar, and (2) applying to the hair a hair protecting composition comprising at least one compound having at least one phosphate group. In an embodiment, step (1) occurs before step (2). In an embodiment, steps (1) and (2) occur simultaneously.

In an embodiment, the compositions may be applied to wet hair. In a further embodiment, method further comprises blow-drying the hair after the composition(s) are applied to the hair. In a further embodiment, method further comprises straightening the hair with heat after the composition(s) are applied to the hair. In a further embodiment, method further comprises curling the hair with heat after the composition(s) are applied to the hair.

A kit according to the disclosure may comprise two or more separately contained components, wherein at least one of the two or more separately contained components is a hair treatment composition comprising: (1) at least one compound comprising at least one phosphate, (2) at least one sugar, and (3) a cosmetically acceptable carrier. In another embodiment, a kit according to the disclosure may comprise two or more containers, wherein a first container comprises (1) a hair protecting composition comprising at least one sugar, and a second container comprises (2) a hair protecting composition comprising at least one compound having at least one phosphate group.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementation of the present technology will now be described, by way of example only, with reference to the attached figures, wherein:

FIG. 1. photos of solutions stored at 45° C. for approximately two months with “browning” as a visual indicator of reaction onset. The initially clear compositions darken (referred to as “browning” or turning “brown”) and appear as varying shades of yellow and brown.

FIG. 2. compares the Denaturation temperatures (Td) of treated swatches depicting a synergistic effect of the combination of xylose and a catalytic quantity of MSP.

FIG. 3. compares the Denaturation temperatures (Td) of treated swatches depicting a synergistic effect of the combination of xylose and phytic acid.

FIG. 4. compares the Denaturation temperatures (Td) of treated swatches depicting a synergistic effect of the combination of xylose and glycerol phosphate.

FIG. 5. compares the Denaturation temperatures (Td) of treated swatches depicting a synergistic effect of the combination of glucose and MSP.

FIG. 6. compares the Denaturation temperatures (Td) of treated swatches depicting a synergistic effect of the combination of Xylose and catalytic concentration of MSP to prevent heat damage.

FIG. 7. compares the Denaturation temperatures (Td) of treated swatches depicting a synergistic effect of the combination of Xylose and Glycerol Phosphate to prevent heat damage.

FIG. 8. compares the Denaturation temperatures (Td) of treated swatches depicting a synergistic effect of the combination of Glucose and MSP to prevent heat damage.

It should be understood that the various aspects are not limited to the arrangements and instrumentality shown in the drawings.

### DESCRIPTION

The disclosure relates to compositions and methods for protecting hair. The compositions comprise synergistic combinations of phosphate-containing compounds and sugars. The methods comprise applying the compositions to the hair for treating the hair or for protecting the hair from heat-induced damage or for reducing heat-induced damage to hair, e.g. from heat applied by heating tools.

I. Compositions

The compositions according to the disclosure comprise unexpectedly synergistic combinations of phosphate-containing compounds and sugars, which surprisingly protect hair from heat and/or reduce damage from heat to hair.

### Sugars

Compositions according to the disclosure comprise at least one sugar. The term “sugar” as used herein is intended to mean oxygen-bearing hydrocarbon-based compounds containing several alcohol functions, with or without aldehyde or ketone functions, and which comprise at least 4 carbon atoms. These sugars may be monosaccharides, disaccharides, oligosaccharides or polysaccharides, and derivatives thereof, such as alkyl derivatives, such as methyl derivatives, for instance methylglucose.

In an embodiment, the sugars are chosen from C3-C6 monosaccharides.

In an embodiment, the sugars are chosen from pentoses and derivatives thereof. In a further embodiment, the pentoses are chosen from xylose, arabinose, ribose, 2-deoxy-ribose, ribulose, deoxy-ribulose, arabinose, xylulose, or mixtures thereof.

In an embodiment, the sugars are chosen from hexoses and derivatives thereof, such as amino hexoses. In a further embodiment, the hexoses are chosen from allose, altrose, glucose (including dextrose), glucosamine, mannose, gulose, idose, galactose, talose, sorbose, psicose, fructose, tagatose, or mixtures thereof.

In an embodiment, the sugars are chosen from disaccharides (which are saccharides that hydrolyze into two monosaccharides). In a further embodiment, the disaccharides are chosen from sucrose (also saccharose), maltose, lactose, cellobiose, trehalose, dextran, or mixtures thereof.

In an embodiment, the sugars are chosen from polysaccharides (which are saccharides that hydrolyze into more than two monosaccharides). In a further embodiment, the polysaccharides are chosen from maltotriose, starch, dextrins, cellulose, glycogen, or mixtures thereof.

In an embodiment, the sugars may be chosen from any aldoses or ketoses or tetroses or teioses or aldotetroses (such as erythrose and treose), ketotetroses (such as erythrulose), aldotrioses (such as glyceraldehyde) and ketotrioses (such as dihydroxyacetone), or furanoses, N-acetyl-D-galactosamine, N-acetyl-D-glucosamine, N-acetylneuraminic acid, adonitol, beta-D-allose, D-altrose, 6-amino-6-deoxy-D-glucose, 1,6-anhydro-glucose, arabinic acid, arabinogalactan, D-arabinose, L-arabinose, D,L-arabinose, D-arabitol, L-arabitol, D-cellobiose, D-glucosamine, D-galactosamine, 2-deoxy-D-glucose, 6-deoxy-D-galactose, 6-deoxy-L-galactose, galactitol, mesoerythritol, D-erythroe, D-fructose, D-fucose, L-fucose, D-galactaric acid, galactitol, galactomannan, D-galactono-1,4-lactone, L-galactono-1,4-lactone, D-galactosarmine, D-galactose, L-galactose, D-galacturonic acid, beta-gentiobiose, glucamine, D-glucaric acid, D-glucono-1,5-lactone, L-glucono-1,5-lactone, D-glucosam ine, D-glucosaminic acid, D-glucuronic acid, L-glucose, D-glucose, isomaltitol, isomaltotriose, isomaltose, lactobionic acid, D-lactose, lactulose, D-lyxose, L-lyxose, lyxosamine, maltitol, D-maltose, maltotetraose, maltotriitol, maltotriose, D-mannosamine, D-mannose, L-mannose, D-melezitose, D-melibiose, D-raffinose, D-raffinose undecaacetate, L-rhamnose, D-ribose, L-ribose, D-ribulose, rutinose, D-sucrose, alpha-sophorose, sorbitol, D-tagatose, D-talose, D-threose, turanose, D-xylitol, D-xylose, L-xylose and D,L-xylose.

Exemplary and non-limiting sugars that can be used include, but are not limited to, xylose, glucose, or combinations thereof.

The total amount of the at least one sugar may vary, but typically is up to about 15%, up to about 10%, up to about 9%, up to about 8%, up to about 7%, up to about 6%, up to about 5%, up to about 4.5%, up to about 4%, up to about 3%, up to about 2%, up to about 1%, relative to the total weight of the composition. The total amount of the at least one sugar may range from about 0.001% to about 15%, about 0.01% to about 10%, about 0.01% to about 5%, about 0.4% to about 4.5%, or about 0.2% to about 2%, relative to the total weight of the composition, including ranges and sub-ranges there between. For example, the total amount of the at least one sugar may range from about 0.01% to about 10%, such as from about 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.01% to about 0.75%, about 0.01% to about 0.5%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.75%, about 0.05% to about 0.5%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.75%, about 0.1% to about 0.5%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or about 5% to about 6%, including all ranges and sub-ranges there between, relative to the total weight of the composition. In various embodiments, the at least one sugar may be present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%, relative to the total weight of the composition, including ranges and sub-ranges there between.

In one embodiment, the sugar is present in an amount of less 6% by weight, relative to the total weight of the composition. In an embodiment, the sugar is present in an amount of less than exactly 6%, such as in an amount up to exactly 5.9999% by weight, relative to the total weight of the composition.

### Phosphates

Compositions according to the disclosure comprise at least one compound having at least one phosphate group, also referred to as a phosphate-containing compound.

Exemplary and non-limiting phosphate compounds that can be used include: alkali metal phosphates such as potassium phosphate, potassium dihydrogen phosphate, dipotassium hydrogen phosphate, sodium phosphate (also referred to as “monosodium phosphate” or “MSP”), sodium dihydrogen phosphate, disodium hydrogen phosphate, calcium phosphate, magnesium phosphate, zinc phosphate; glycerol phosphate (or “glyceryl phosphate” or “glycerophosphate”), phytic acid, as well as combinations thereof. Glycerol or glyceryl phosphate may be available as a metal phosphate such as disodium glycerol phosphate (or sodium glycerol phosphate and commercially available from the company SEPPIC), calcium glycerol phosphate, magnesium glycerol phosphate, disodium diglyceryl monophosphate, or aluminum isostearyl glyceryl phosphate.

Polyphosphorous derivatives may be chosen from inorganic polyphosphates, preferably chosen from: optionally hydrated alkali metal pyrophosphates, preferably chosen from sodium pyrophosphate, potassium pyrophosphate, sodium pyrophosphate decahydrate; and polymetaphosphates, such as sodium hexametaphosphate, sodium trimetaphosphate; sodium polyphosphates such as sodium tripolyphosphate, and mixtures thereof. Sodium and/or potassium is preferably used as alkali metal. According to another embodiment, polyphosphorous derivatives are chosen from organic polyphosphorous derivatives, preferably from polyphosphoric acids and/or salts thereof, such as phytic acid, polyphosphonic acids and/or salts thereof, such as EDTMP, DETMP, ATMP, HEDP, DTPMP, and mixtures thereof.

The total amount of the at least one compound having at least one phosphate group may vary, but typically is up to about 15%, such as up to about 10%, up to about 9%, up to about 8%, up to about 7%, up to about 6%, up to about 5%, up to about 4.5%, up to about 4%, up to about 3%, up to about 2%, up to about 1%, relative to the total weight of the composition. The total amount of the at least one compound having at least one phosphate group may range from about 0.001% to about 15%, such as about 0.01% to about 10%, about 0.01% to about 5%, about 0.4% to about 4.5%, or about 0.2% to about 2%, relative to the total weight of the composition, including ranges and sub-ranges there between. For example, the total amount of the at least one compound having at least one phosphate group may range from about 0.001% to about 10%, such as from about 0.001% to about 9%, about 0.001% to about 8%, about 0.001% to about 7%, about 0.001% to about 6%, about 0.001% to about 5%, about 0.001% to about 4%, about 0.001% to about 3%, about 0.001% to about 2%, about 0.001% to about 1%, about 0.001% to about 0.75%, about 0.001% to about 0.5%, about 0.001% to about 0.4%, about 0.001% to about 0.3%, about 0.001% to about 0.2%, about 0.001% to about 0.1%, about 0.001% to about 0.09%, about 0.001% to about 0.08%, about 0.001% to about 0.07%, about 0.001% to about 0.06%, about 0.001% to about 0.05%, 0.01% to about 9%, about 0.01% to about 8%, about 0.01% to about 7%, about 0.01% to about 6%, about 0.01% to about 5%, about 0.01% to about 4%, about 0.01% to about 3%, about 0.01% to about 2%, about 0.01% to about 1%, about 0.01% to about 0.75%, about 0.01% to about 0.5%, about 0.01% to about 0.4%, about 0.01% to about 0.3%, about 0.01% to about 0.2%, about 0.01% to about 0.1%, about 0.01% to about 0.09%, about 0.01% to about 0.08%, about 0.01% to about 0.07%, about 0.01% to about 0.06%, about 0.01% to about 0.05%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.75%, about 0.05% to about 0.5%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.75%, about 0.1% to about 0.5%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or about 5% to about 6%, including all ranges and sub-ranges there between, relative to the total weight of the composition. In various embodiments, the at least one compound having at least one phosphate group may be present in an amount of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10%, relative to the total weight of the composition, including ranges and sub-ranges there between.

When the at least one compound having at least one phosphate group in the compositions according to the disclosure includes monosodium phosphate, the total amount of monosodium phosphate may range from about 0.001% to about 2%, such as from about 0.001% to about 1.5%, about 0.001% to about 1%, about 0.001% to about 0.75%, about 0.001% to about 0.5%, about 0.001% to about 0.4%, about 0.001% to about 0.3%, about 0.001% to about 0.2%, about 0.001% to about 0.1%, about 0.001% to about 0.09%, about 0.001% to about 0.08%, about 0.001% to about 0.07%, about 0.001% to about 0.06%, about 0.001% to about 0.05%, relative to the total weight of the composition, including ranges and sub-ranges there between.

When the at least one compound having at least one phosphate group in the compositions according to the disclosure includes phytic acid, the total amount of phytic acid may range from about 0.001% to about 10%, such as from about 0.001% to about 9%, about 0.001% to about 8%, about 0.001% to about 7%, about 0.001% to about 6%, about 0.001% to about 5%, about 0.001% to about 4%, about 0.001% to about 3%, about 0.001% to about 2%, about 0.001% to about 1%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.75%, about 0.05% to about 0.5%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.75%, about 0.1% to about 0.5%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or about 5% to about 6%, including all ranges and sub-ranges there between, relative to the total weight of the composition, relative to the total weight of the composition, including ranges and sub-ranges there between.

When the at least one compound having at least one phosphate group in the compositions according to the disclosure includes glycerol phosphate, the total amount of glycerol phosphate may range from about 0.001% to about 10%, such as from about 0.001% to about 9%, about 0.001% to about 8%, about 0.001% to about 7%, about 0.001% to about 6%, about 0.001% to about 5%, about 0.001% to about 4%, about 0.001% to about 3%, about 0.001% to about 2%, about 0.001% to about 1%, about 0.05% to about 10%, about 0.05% to about 9%, about 0.05% to about 8%, about 0.05% to about 7%, about 0.05% to about 6%, about 0.05% to about 5%, about 0.05% to about 4%, about 0.05% to about 3%, about 0.05% to about 2%, about 0.05% to about 1%, about 0.05% to about 0.75%, about 0.05% to about 0.5%, about 0.1% to about 10%, about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to about 1%, about 0.1% to about 0.75%, about 0.1% to about 0.5%, about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, or about 5% to about 6%, including all ranges and sub-ranges there between, relative to the total weight of the composition, relative to the total weight of the composition, including ranges and sub-ranges there between.

Optionally, the weight ratio of the total amount of compound having at least one phosphate group to the sugar may be chosen to range from about 1:100 to about 100:1, about 1:100 to about 100:1, about 1:50 to about 50:1, about 1:40 to about 40:1, about 1:30 to about 30:1, about 1:25 to about 25:1, about 1:20 to about 20:1, about 1:15 to about 15:1, about 1:10 to about 10:1, about 1:8 to about 8:1, about 1:7 to about 7:1, about 1:6 to about 6:1, about 1:5 to about 5:1, about 1:4.5 to about 1:4.5, about 1:4 to about 1:4, about 1:3.5 to about 1:3.5, about 1:3 to about 1:3, about 1:2.5 to about 1:2.5, about 1:2 to about 1:2, about 1:1.5 to about 1:1.5, including ranges and sub-ranges there between. Optionally, the weight ratio of the total amount of compound having at least one phosphate group to the sugar may be about 1:100, about 1:50, about 1:25, about 1:15, about 1:10, about 1:9, about 1:8, about 1:7, about 1:6, about 1:5, about 1:4.5, about 1:4, about 1:3.5, about 1:3, about 1:2.5, about 1:2, about 1:1.5, about 1:1, about 1:0.9, about 1:0.8, about 1:0.7, about 1:0.5, about 100:1, about 50:1, about 25:1, about 15:1, about 10:1, about 9:1, about 8:1, about 7:1, about 6:1, about 5:1, about 4.5:1, about 4:1, about 3.5:1, about 3:1, about 2.5:1, about 2:1, about 1.5:1, about 0.9:1, about 0.8:1, about 0.7:1, or about 0.5:1, including ranges and sub-ranges there between.

### pH Adjusters

It may, in at least certain embodiments, be beneficial to adjust the pH of the compositions. By way of example only, in an embodiment, the pH of the disclosed compositions may advantageously be at or below about 8.5, such as at or below about 8.0, or at or below about 7.5. For example, the pH may be below 7.5.

Compositions according to the disclosure may optionally also contain acid and alkali pH adjusters (also modifiers). Such pH adjusters include, but are not limited to, sodium hydroxide, hydrochloric acid, lactic acid, sodium metasilicate, silicate compounds, citric acid, ascorbic acid, and carbonate compounds. The disclosed compositions may also be substantially free of acid and alkali pH adjusters.

Non-limiting examples of acidic pH modifiers include organic acids and/or inorganic acids. In some instances, the compositions include at least one non-polymeric mono, di, and/or tricarboxylic acid. A non-polymeric mono, di, and/or tricarboxylic acid is an organic compound having one (mono), two (di), or three (tri) carboxylic acid groups (—COOH). The non-polymeric mono, di, and tricarboxylic acids, and/or salts thereof, typically have a molecular weight of less than about 500 g/mol, less than about 400 g/mol, or less than about 300 g/mol.

Non-limiting examples of monocarboxylic acids, or salts thereof, include formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, salts thereof, and a mixture thereof. In some cases, the hair-treatment compositions include at least lactic acid and/or acetic acid, and/or salts thereof.

Non-limiting examples of dicarboxylic acids and/or salts thereof include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, salts thereof, and a mixture thereof. In some cases, the hair-treatment compositions include oxalic acid, malonic acid, malic acid, maleic acid, salts thereof, or a mixture thereof. Non-limiting examples of tricarboxylic acids and salts thereof include citric acid, isocitric acid, aconitric acid, propane-1,2,3-tricarboxylic acid, benzene-1,3,5-tricarboxylic acid, salts thereof, and mixtures thereof. In some instances, the hair-treatment compositions include at least citric acid and/or salts thereof.

In some instances, the hair-treatment composition include one or more inorganic pH modifiers. Non-limiting examples include hydrochloric acid, phosphoric acid, sulfuric acid, hydrobromic acid, hydriodic acid, phosphoric acid, boric acid, etc.

Without wishing to be bound by theory, it is believed that the phosphate acts as a catalyst to change the sugar structure to increase reactivity and a higher pH increases the reaction rate for a given set of solutions by making the amine more nucleophilic and therefore more reactive.

It may, in at least certain embodiments, be beneficial to adjust the pH of the compositions. By way of example only, in an embodiment, the pH of the disclosed compositions may be at or below about 8.5, 8, 7.5, 7, 6.5, 6, 5.5, 5, 4.5, 4, 3.5, 3, or 2.5. In an embodiment, the pH of the disclosed compositions may range from about 8.5 to about 2, from about 8 to about 2, from about 7.5 to about 2, from about 7.5 to about 2.5, from about 8.5 to about 2.5, from about 8 to about 2.5, from about 7.5 to about 2.5, from about 7.5 to about 3, from about 7.5 to about 3.5, from about 7.5 to about 4, from about 7.5 to about 4.5, from about 7.5 to about 5, from about 7.5 to about 5.5, from about 7.5 to about 6, or from about 7.5 to about 6.5, including ranges and sub-ranges there between.

### Solvents

In certain exemplary embodiments, compositions according to the disclosure comprise at least one physiologically acceptable medium. The physiologically acceptable medium may be chosen from water or a mixture of water and at least one cosmetically acceptable solvent. Non-limiting examples of cosmetically acceptable solvents include C2-C4 lower alcohols, such as ethanol and isopropanol; polyols, especially those containing from 2 to 6 carbon atoms, for instance glycerol, propylene glycol, butylene glycol, pentylene glycol, hexylene glycol, dipropylene glycol or diethylene glycol; polyol ethers, for instance 2-butoxyethanol, propylene glycol monomethyl ether and diethylene glycol monomethyl ether or monoethyl ether; and mixtures thereof.

In one exemplary embodiment, the composition comprises a solvent or solvent mixture in an amount up to about 99.9%, such as ranging from about 50% to about 99%, about 55% to about 95%, or about 60% to about 90%, by weight relative to the weight of the composition. For example, the composition may comprise water in an amount up to about 99%, such as, for example, an amount ranging from about 50 to about 95%, or from about 60% to about 90%, by weight relative to the weight of the composition.

### Additional Components

The compositions according to the disclosure may optionally also comprise additives useful or desired for preparing hair protecting compositions. For example, it may be beneficial to include components that will aid in application of the composition, such as glycerol. Exemplary and non-limiting additives include nacreous agents, dyes or pigments, fragrances, mineral, plant or synthetic oils, waxes, vitamins, proteins including ceramides, vitamins, UV-screening agents, free-radical scavengers, antidandruff agents, hair-loss counteractants, hair restorers, preserving agents, and mixtures thereof. A person skilled in the art will take care to select the optional additives and the amount thereof such that they do not harm the properties of the compositions of the present disclosure.

The compositions of certain embodiments may comprise stabilizers, for example sodium chloride, magnesium dichloride or magnesium sulfate.

The compositions according to the disclosure may additionally comprise cosmetic adjuvants chosen from fragrances, pigments, chelating agents, softeners, antioxidants, opacifiers, stabilizers, moisturizing agents, vitamins, bactericides, preservatives, polymers, thickening agents, or any other ingredient commonly used in cosmetics for this type of application.

In certain embodiments, these additives are generally present in an amount ranging up to about 40% by weight of active material relative to the total weight of the composition, such as up to about 30%, up to about 20%, up to about 15%, up to about 10%, up to about 5%, such as from 0.01% to 30%.

In an embodiment, the compositions may be substantially free of reducing agents, such as thiol based reducing agents.

The compositions described herein may be in any suitable physical form. Suitable forms include, but are not limited to, low to moderate viscosity liquids, lotions, milks, gel creams, creams, pastes, clays, conditioners, masks, sprays, and the like.

If desired, a person of skill in the art can select the additives or amounts thereof in order to maintain the desired properties of the compositions. The compositions may be packaged in a variety of different containers, such as, for example, a ready-to-use container. Non-limiting examples of useful packaging include tubes, jars, caps, unit dose packages, and bottles, including squeezable tubes and bottles, bottles configured with pump dispensers, and spray bottles.

I. Methods

Methods according to the disclosure comprise applying compositions described herein to the hair in order to treat the hair, to protect the hair from heat-induced damage, or to reduce damage to the hair. In an embodiment, the methods involve protecting hair or minimizing/reducing heat-induced damage to hair, the method comprising applying compositions described herein to the hair. The methods can also repair hair. In an exemplary embodiment, the composition is a leave-in hair protecting composition.

In an embodiment, the methods comprise applying to the hair a hair protecting composition comprising at least one compound comprising at least one phosphate and a composition comprising at least one sugar, as described herein. The compositions may be applied simultaneously or consecutively. The composition comprising the sugar may be applied before or after the composition comprising the compound comprising at least one phosphate. The compound comprising at least one phosphate and sugar can be pre-mixed in one composition prior to application to hair. In an embodiment, the methods comprise applying to the hair a hair protecting composition comprising at least one compound comprising at least one phosphate and at least one sugar.

Methods of treating hair according to the disclosure may include applying a composition according to disclosure to hair (wet, damp, or dry hair). The composition may be applied to the hair before, during, or after any hair styling, shaping, and/or caring process.

An individual may choose to heat the hair before, during, or after the composition is applied to the hair, such as, for example, using a hair dryer (or blow dryer) and/or flat iron. In an embodiment, the composition may be applied to the hair before a heat-induced styling or shaping process, such as blow-drying, curling using a curling iron, flat iron, hot curlers, curlers with blow drying, a heating hood or blowing out using a hair dryer, or straightening the hair using a flat iron or blowing out with a hair dryer. The composition may be applied to the hair before, with, or after other hair compositions (e.g., a shampoo, a conditioner, a mask, a cream, a lotion, a gel, a spray, a styling product, etc.). The methods may comprise heating the hair, optionally with a device having a temperature of about 40° C. to about 210° C. An individual may choose to heat the styled hair before, during, or after the composition is applied to the hair, such as, for example, using a hair dryer and/or flat iron. In an embodiment, the composition may be applied to the hair before a heat-induced styling process, such as blow-drying; curling using a curling iron, flat iron, hot curlers, curlers with blow drying, or blowing out using a hair dryer, or straightening the hair using a flat iron or blowing out with a hair dryer. In an embodiment, the hair is heated using a device having a temperature of at least about 20° C., 30° C., 40° C., 50° C., 60° C., 70° C., 80° C., 90° C., 100° C., 110° C., 120° C., 130° C., 140° C., 150° C., 160° C., 170° C., 180° C., 190° C., 200° C., 210° C., 220° C. In an embodiment, the hair is heated using a device having a temperature ranging from about 20° C. to about 220° C., 20° C. to about 130° C., about 30° C. to about 120° C., about 40° C. to about 110° C., about 50° C. to about 100° C., about 60° C. to about 90° C., about 70° C. to about 80° C., about 60° C. to about 220° C., about 70° C. to about 210° C., about 80° C. to about 200° C., about 90° C. to about 190° C., about 100° C. to about 180° C., about 110° C. to about 170° C., about 120° C. to about 160° C., about 130° C. to about 150° C. The composition may be applied to the hair before, with, or after other hair compositions (e.g., a shampoo, a conditioner, a mask, a cream, a lotion, a gel, a spray, etc.).

In various embodiments, the hair is dried or heated using a device having a temperature ranging from room temperature up to at least 40° C., for example, using a blow dryer, while optionally applying a smoothing action on the hair. The smoothing action may be conducted by brushing or combing or passing the fingers through the hair.

Drying the hair using a device having a temperature ranging from room temperature up to at least 40° C. may be accomplished by drying the hair with blow dryer device or using other heat sources such as a flat iron, a hair dryer, a heat lamp, a heat wand, or other similar devices. When a blow dryer is used, the blow dryer may be employed at a temperature ranging from about 30° C. to about 90° C. or from about 30° C. to about 60° C. or from about 35° C. to about 60° C., including ranges and sub-ranges therebetween.

When a flat iron is used, the flat iron may be employed at a temperature of about 100° C. or more, such as ranging from about 100° C. to about 250° C., from about 110° C. to about 230° C., from about 110° C. to about 210° C., from about 120° C. to about 200° C., from about 150° C. to about 190° C., or from about 190° C. to about 230° C., including ranges and sub-ranges there between, or at a temperature of about 230° C., about 225° C., about 220° C., about 210° C., about 200° C., about 190° C., about 180° C., about 150° C., or about 100° C., including ranges and sub-ranges therebetween, and preferably at about 230° C. In various embodiments, the flat iron is passed over the hair at least 1, 2, 3, 4, 5, 6, 7, 8, 9, 10 times or more, for example, up to 20, 30, 40, 50, 75, or 100 times.

Kits according to the disclosure may include two or more separately contained components, wherein at least one of the two or more separately contained components is a hair treatment composition comprising: (1) at least one compound comprising at least one phosphate, (2) at least one sugar, and a cosmetically acceptable carrier. In further embodiments, kits according to the disclosure may comprise two or more containers, wherein a first container comprises (1) a hair protecting composition comprising at least one sugar, and a second container comprises (2) a hair protecting composition comprising at least one compound having at least one phosphate group.

It is to be understood that although compositions and/or processes according to the disclosure generally demonstrate one or more of the properties described herein, compositions according to the disclosure may not demonstrate all or some of the disclosed properties, yet the compositions and methods of protecting hair are still within the scope of the disclosure.

The following definitions are provided for the present disclosure only.

As used herein, the term “hair” is meant to include only hair on the scalp of the head, and does not include eyelashes or eyebrows.

The terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

The terms “a,” “an,” “the,” and “at least one” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” “2% to 5%,” and so on. Unless expressly indicated otherwise, all numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Unless otherwise indicated, all percentages herein are by weight, relative to the weight of the total composition.

The term “substantially free” as used herein means the specific material may be present in small amounts that do not materially affect the basic and novel characteristics of the compositions according to the disclosure. For instance, there may be less than 2% by weight of a specific material added to a composition, relative to the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free” or “substantially free” of one or more components that are positively set forth in the instant disclosure.

The term “substantially free” as used herein may also mean that the specific material is not added to the composition but may still be present in a raw material that is included in the composition.

“Cosmetic composition” encompasses many types of compositions for application to hair, for example, hair lotions, hair creams, hair gel creams, hair conditioners, hair masques (masks), etc. In an embodiment, the compositions are leave-on hair compositions.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

### EXAMPLES

The following examples are intended to be non-limiting and explanatory in nature only.

### Example 1

Solutions stored at 45° C. were visually inspected to evaluate the impact of phosphate and pH on reaction kinetics between a sugar (xylose) and an amine (threonine, to represent the amines available in hair protein) by observing the onset of color change for each exemplified solution.

The following hair protecting compositions were prepared by mixing the components, as disclosed in Table 1. The solution's pH was adjusted to 7.5 as necessary using sodium hydroxide. Amounts are expressed in wt % of active materials:

TABLE 1

| | Composition | | | |
|---|---|---|---|---|
| | 1A | 1B | 1C | 1D |
| Xylose | 1 | 1 | 1 | 1 |
| Threonine | 0.8 | 0.8 | 0.8 | 0.8 |
| Monosodium | — | 0.8 | — | 0.8 |
| phosphate | | | | |
| pH | 5.67 (not | 4.56 (not | 7.5 | 7.5 |
| | adjusted) | adjusted) | (adjusted) | (adjusted) |
| DI water | Q.S. 100 | Q.S. 100 | Q.S. 100 | Q.S. 100 |
| Browning | ~2 months | ~2 weeks | ~3 weeks | 1 day |
| Onset | | | | |

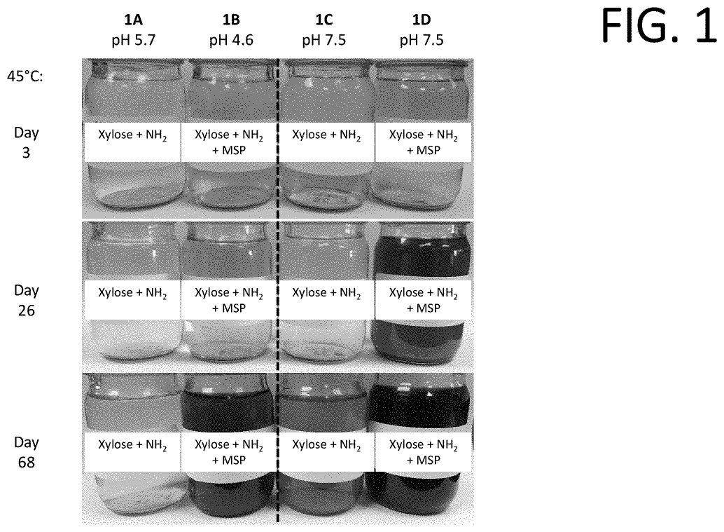

Photos of the composition were taken at three time points (day 3, day 26, and day 68) over the course of approximately two months, and changes in color of compositions 1A-1D from the initial clear, uncolored solutions were noted. Browning (initially seen as yellowing) was a visual indicator of the Maillard reaction occurring. As shown in FIG. 1, addition of monosodium phosphate in compositions 1B and 1D surprisingly increased the reaction rate for both high and low pH solutions. The relative reaction rate of each composition was ranked as follows: 1D>1B>1C>1A [+MSP 7.5>+MSP 4.56>−MSP 7.5>−MSP 5.67].

### Example 2

Swatch studies were performed on double bleached swatches of hair (bleached about one week prior) and natural hair swatches to evaluate the impact of the disclosed compositions on the denaturation temperature of hair as measured by Differential Scanning calorimetry (DSC).

For the one-step composition application methods, double bleached hair or natural hair swatches were washed, dabbed dry, and each treated with one of the exemplified compositions or a mixture of the exemplified compositions in Example 2 by applying 0.4 g of the composition/gram of hair with a pipette and working it in with gloved hands and a comb. After waiting 5 minutes, each swatch was roughly blow dried for 120 seconds then brush blow dried 10 passes. The swatches were then flat ironed for 3 passes at 190° C. using a comb in between to align fibers.

For the two-step composition application methods, double bleached hair or natural hair swatches were washed, dabbed dry, and each treated with one of the exemplified sugar compositions in Example 2 by applying 0.4 g of the composition/gram of hair with a pipette and working it in with gloved hands and a comb. After waiting 2.5 minutes, the swatches were further treated with one of the exemplified phosphate compositions in Example 2 by applying 0.4 g of the composition/gram of hair with a pipette and working it in with gloved hands and a comb. After waiting 2.5 minutes, each swatch was roughly blow dried for 120 seconds then brush blow dried 10 passes. The swatches were then flat ironed for 3 passes at 190° C. using a comb in between to align fibers.

Thermal Stress Tests were performed on only the natural hair swatches. The thermal stress tests involved flat ironing the swatches for 5 passes at 232° C. at a speed of 12 seconds/pass (half the typical flat iron application speed) using comb in between to align fibers. Then waiting at least 5 minutes for swatch to cool down before flat ironing the swatch for an additional 5 passes to reach a total of 10 passes.

Differential Scanning calorimetry (DSC) was performed on the treated double bleached hair swatches and the natural hair swatches. About 8 mg of finely cut hair taken from the middle portion of the swatch (not including tip and root sections) was subjected to DSC. DSC was then performed on cut hair from each swatch with 50 μL water added to a sealed pan. The sealed pan containing the hair mixed in water sat overnight at room temperature (˜16 hours) before being run in the DSC instrument. The samples were heated at a ramp rate of 10° C./min to 180° C. The data was analyzed to determine the denaturation temperature (Td) of each swatch. A higher Td value means that more heat is required to denature the keratin protein.

Set 1

The following hair protecting compositions were prepared by mixing the components, as disclosed in Table 2. All the solutions had a pH of 7.5 (pH adjusted as necessary with NaOH). Amounts are expressed in wt % of active materials:

TABLE 2

| | Composition | | | | | |
|---|---|---|---|---|---|---|
| | 2A | 2B | 2C | 2D | 2E | 2F |
| Xylose | — | 0.5 | 1 | — | 0.5 | 1 |
| Monosodium | — | — | — | 0.5 | 0.5 | 0.5 |
| Phosphate | | | | | | |
| DI water | Q.S. 100 | Q.S. 100 | Q.S. 100 | Q.S. 100 | Q.S. 100 | Q.S. 100 |

Compositions 2A to 2F were applied to separate double bleached hair swatches as described above in Example 2. Finally, composition 2B (containing 0.5% xylose) and composition 2C (containing 1% xylose) were applied to two additional, separate swatches, and composition 2D (containing 0.5% MSP) was applied to both swatches in a second consecutive step using the two-step method of Example 2. The swatches were subjected to DSC.

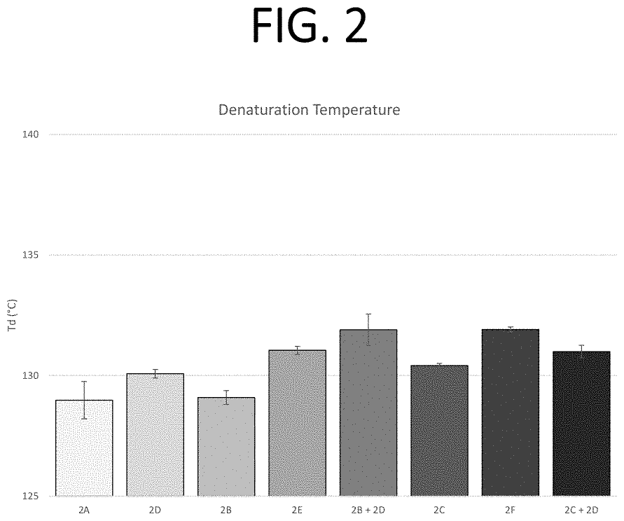

As shown in FIG. 2, the combination of Xylose (0.5 and 1%) and MSP, using either the one-step and two-step application method, imparted statistically higher Td values to hair compared to the DI water controls. The relative increase (improvement) in Td values imparted by each composition was determined to be as follows: xylose+MSP combinations>xylose 1%>MSP 0.05%>xylose 0.5%˜DI water. (“˜” meaning “approximately equal” or “similar” throughout the Examples.)

While the treatment of hair with the compositions containing either sugar or phosphates alone resulted in higher denaturation temperatures of the hair compared to the DI water controls, surprisingly and unexpectedly the combination of sugar and phosphate-containing compounds (in the one-step or two-step methods) synergistically increased or improved the denaturation temperature of the treated hair swatches. Such results demonstrate that the compositions protect hair from damaging effects of heat.

Set 2

The following hair protecting compositions were prepared by mixing the components, as disclosed in Table 3. All the solutions had a pH of 7.5 (pH adjusted as necessary with NaOH). Amounts are expressed in wt % of active materials:

TABLE 3

| | Composition | | | |
|---|---|---|---|---|
| | 3A | 3B | 3C | 3D |
| Xylose | — | 1 | — | 1 |
| Phytic Acid | — | — | 4.4 | 4.4 |
| DI water | Q.S. 100 | Q.S. 100 | Q.S. 100 | Q.S. 100 |

Compositions 3A to 3D were applied to separate double bleached hair swatches as described above in Example 2. Finally, composition 3B (containing 1 xylose) was applied to an additional separate swatch, and composition 3C (containing 4.4% phytic acid) was applied to the same swatch in a second consecutive step using the two-step method of Example 2. The swatches were subjected to DSC.

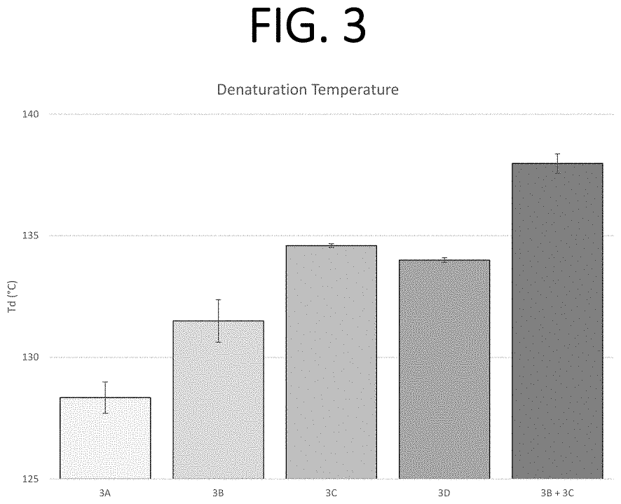

As shown in FIG. 3, the Xylose 1%+Phytic Acid 4.4% two-step application swatch had a statistically higher Td value compared to the controls (compositions 3A-3C alone). The relative increase (improvement) in Td values imparted by each composition was determined to be as follows: xylose+phytic acid two-step combination>phytic acid>xylose+phytic acid one-step combination>xylose>DI water.

While the treatment of hair with the compositions containing either sugar or phosphates alone resulted in higher denaturation temperatures of the hair compared to the DI water controls, surprisingly and unexpectedly the combination of sugar and phosphates (in the one step or two step methods) synergistically increased or improved the denaturation temperature of the treated hair swatches. Such results demonstrate that the compositions protect hair from damaging effects of heat.

Set 3

The following hair protecting compositions were prepared by mixing the components, as disclosed in Table 4. All the solutions had a pH of 7.5 (pH adjusted as necessary with either NaOH or lactic acid). Amounts are expressed in wt % of active materials:

TABLE 4

| | Composition | | | |
|---|---|---|---|---|
| | 4A | 4B | 4C | 4D |
| Xylose | — | 1 | — | 1 |
| Glycerol | — | — | 4.32 | 4.32 |
| Phosphate | | | | |
| DI water | Q.S. 100 | Q.S. 100 | Q.S. 100 | Q.S. 100 |

Compositions 4A to 4D were applied to separate double bleached hair swatches as described above in Example 2. Finally, composition 4B (containing 1 xylose) was applied to an additional separate swatch, and composition 4C (containing 4.32% glycerol phosphate) was applied to the same swatch in a second consecutive step using the two-step method of Example 2. The swatches were subjected to DSC.

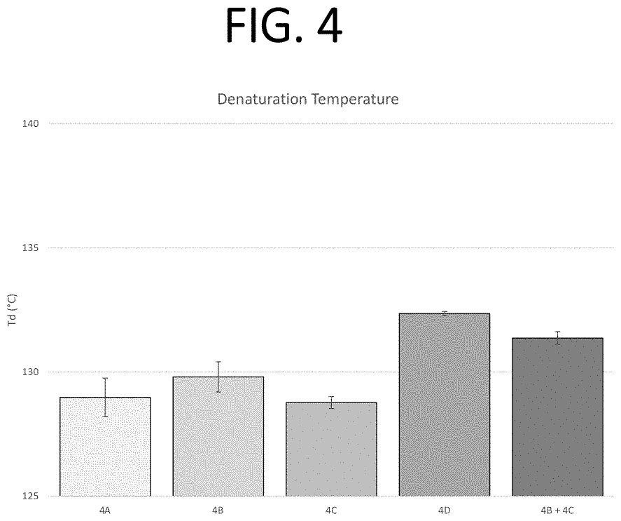

As shown in FIG. 4, the Xylose 1%+Glycerol Phosphate 4.32% one-step and two-step application swatches had a statistically higher Td value compared to the controls (compositions 4A-4C alone). The relative increase (improvement) in Td values imparted by each composition was determined to be as follows: xylose+glycerol phosphate one-step combination>xylose+glycerol phosphate two-step combination>xylose˜DI water˜glycerol phosphate.

While the treatment of hair with the compositions containing either sugar or phosphates alone resulted in higher denaturation temperatures of the hair compared to the DI water controls, surprisingly and unexpectedly the combination of sugar and phosphates (in a one step or two step method) synergistically increased or improved the denaturation temperature of the treated hair swatches. Such results demonstrate that the compositions protect hair from damaging effects of heat.

Set 4

The following hair protecting compositions were prepared by mixing the components, as disclosed in Table 5. All the solutions had a pH of 7.5 (pH adjusted as necessary with NaOH). Amounts are expressed in wt % of active materials:

TABLE 5

| | Composition | | | | | |
|---|---|---|---|---|---|---|
| | 5A | 5B | 5C | 5D | 5E | 5F |
| Glucose | — | 1 | — | — | 1 | 1 |
| Monosodium | — | — | 0.05 | 0.2 | 0.5 | 0.2 |
| Phosphate | | | | | | |
| DI water | Q.S. 100 | Q.S. 100 | Q.S. 100 | Q.S. 100 | Q.S. 100 | Q.S. 100 |

Compositions 5A to 5F were applied to separate double bleached hair swatches as described above in Example 2. Finally, composition 5B (containing 1 glucose) was applied to two additional separate swatches, and composition 5C (containing 0.05% MSP) or composition 5D (containing 0.2% MSP) was applied to one of the swatches in a second consecutive step using the two-step method of Example 2. The swatches were subjected to DSC.

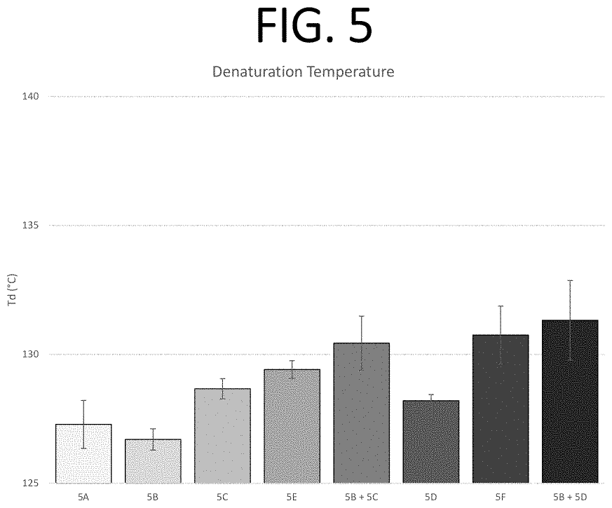

As shown in FIG. 5, the MSP 0.2% or Glucose 1%+MSP combinations (0.05% and 0.2% MSP) (one- and two-step applications) imparted a statistically higher Td value to hair compared to the controls. Glucose at 1% did not increase Td unless phosphate was present. The relative increase (improvement) in Td values imparted by each composition was determined to be as follows: glucose 1%+MSP 0.2% (one- and two-step)˜glucose 1%+MSP 0.05% (one- and two-step)>MSP 0.05%>MSP 0.2%˜DI water˜glucose 1%.

While the treatment of hair with the compositions containing either sugar or phosphates alone resulted in higher denaturation temperatures of the hair compared to the DI water controls, surprisingly and unexpectedly the combination of sugar and phosphates (in a one step or two step method) synergistically increased or improved the denaturation temperature of the treated hair swatches. Such results demonstrate that the compositions protect hair from damaging effects of heat. It is further surprising that the c6 sugar (glucose) provided heat protection, which had not been previously demonstrated.

Set 5

The following hair protecting compositions were prepared by mixing the components, as disclosed in Table 6. All the solutions had a pH of 7.5 (pH adjusted as necessary with NaOH). Amounts are expressed in wt % of active materials:

TABLE 6

| | Composition | | | |
|---|---|---|---|---|
| | 6A | 6B | 6C | 6D |
| Xylose | — | 1 | — | 1 |
| Monosodium | — | — | 0.05 | 0.05 |
| Phosphate | | | | |
| DI water | Q.S. 100 | Q.S. 100 | Q.S. 100 | Q.S. 100 |

The following compositions were applied to one set of swatches: Compositions 6A to 6D were applied to separate natural hair swatches as described above in Example 2. Finally, composition 6B (containing 1% xylose) was applied to an additional separate swatch, and composition 6C (containing 0.05% MSP) was applied to the same swatch in a second consecutive step using the two-step method of Example 2. Snippets for DSC were collected after this treatment, then the swatches were subjected to thermal stress tests, followed by DSC on snippets of the hair.

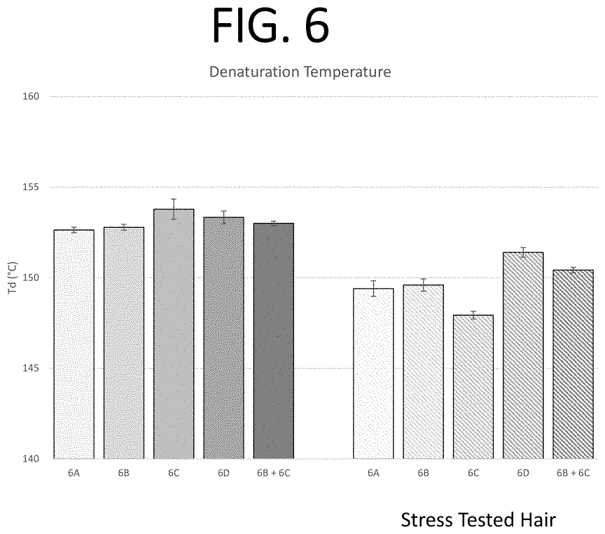

As shown in FIG. 6, the Xylose+MSP combinations (one step and two step) imparted a statistically higher Td value to hair compared to the controls (compositions 6A-6C alone) after 10 slow passes with the flat iron at 232° C. (the thermal stress test). The relative increase (improvement) in Td values imparted by each composition was determined to be as follows: xylose+MSP one step>xylose+MSP two step>xylose˜DI water>MSP.

The compositions comprising sugars and phosphates surprisingly demonstrated beneficial heat protecting properties synergistically in combination with each other, including preventing the decrease of (improving) the denaturation temperature of the treated hair swatches subjected to a thermal stress test, thus demonstrating that the compositions protect hair from heat.

Set 6

The following hair protecting compositions were prepared by mixing the components, as disclosed in Table 7. All the solutions had a pH of 7.5 (pH adjusted as necessary with lactic acid). Amounts are expressed in wt % of active materials:

TABLE 7

| | Composition | | | |
|---|---|---|---|---|
| | 7A | 7B | 7C | 7D |
| Xylose | — | 1 | — | 1 |
| Glycerol | — | — | 4.32 | 4.32 |
| Phosphate | | | | |
| DI water | Q.S. 100 | Q.S. 100 | Q.S. 100 | Q.S. 100 |

The following compositions were applied to one set of swatches: Compositions 7A to 7D were applied to separate natural hair swatches as described above in Example 2. Finally, composition 7B (containing 1% xylose) was applied to an additional separate swatch, and composition 7C (containing 4.32% glycerol phosphate) was applied to the same swatch in a second consecutive step using the two-step method of Example 2. Snippets for DSC were collected after this treatment, then the swatches were subjected to thermal stress tests, followed by DSC on snippets of the hair.

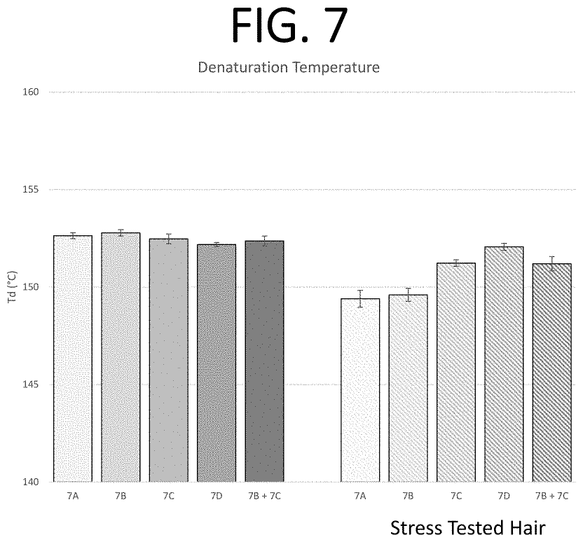

As shown in FIG. 7, the Xylose+Glycerol Phosphate 1 step application combo has a statistically higher Td value compared to the controls (compositions 7A-7C alone) after 10 slow passes with the flat iron at 232° C. (the thermal stress test). The relative increase (improvement) in Td values imparted by each composition was determined to be as follows: xylose+GP one step>xylose+GP two step˜GP>xylose˜DI water.

The compositions comprising sugars and phosphates surprisingly demonstrated beneficial heat protecting properties synergistically in combination with each other, including preventing the decrease of (improving) the denaturation temperature of the treated hair swatches subjected to a thermal stress test, thus demonstrating that the compositions protect hair from heat.

Set 7

The following hair protecting compositions were prepared by mixing the components, as disclosed in Table 8. All the solutions had a pH of 7.5 (pH adjusted as necessary with NaOH). Amounts are expressed in wt % of active materials:

TABLE 8

| | Composition | | | |
|---|---|---|---|---|
| | 8A | 8B | 8C | 8D |
| Glucose | — | 1 | — | 1 |
| MSP | — | — | 0.2 | 0.2 |
| DI water | Q.S. 100 | Q.S. 100 | Q.S. 100 | Q.S. 100 |

The following compositions were applied to one set of swatches: Compositions 8A to 8D were applied to separate natural hair swatches as described above in Example 2. Finally, composition 8B (containing 1% xylose) was applied to an additional separate swatch, and composition 8C (containing 0.2% MSP) was applied to the same swatch in a second consecutive step using the two-step method of Example 2. Snippets for DSC were collected after this treatment, then the swatches were subjected to thermal stress tests, followed by DSC.

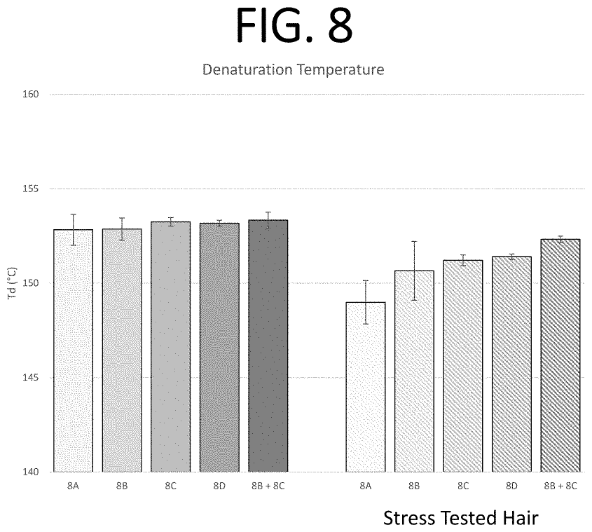

As shown in FIG. 8, the Glucose+MSP 2 step combo imparts a statistically higher Td value compared to the controls (compositions 8A-8C alone) after 10 slow passes with the flat iron at 232° C. (the thermal stress test). The relative increase (improvement) in Td values imparted by each composition was determined to be as follows: glucose+MSP two step>glucose+MSP one step˜MSP˜Glucose>DI water.

The compositions comprising sugars and phosphates surprisingly demonstrated beneficial heat protecting properties synergistically in combination with each other, including preventing the decrease of (improving) the denaturation temperature of the treated hair swatches subjected to a thermal stress test, thus demonstrating that the compositions protect hair from heat.

1-67. (canceled)

68. A hair protecting composition comprising:
a) at least one sugar, and
b) at least one compound having at least one phosphate group;
wherein the at least one sugar is present in an amount of less than 6% by weight, relative to the total weight of the composition.

69. The hair protecting composition of claim 68, wherein the at least one compound having at least one phosphate group is present in an amount of up to about 4.5%, relative to the total weight of the composition.

70. The hair protecting composition of claim 68, wherein the at least one compound having at least one phosphate group is chosen from monosodium phosphate, phytic acid or glycerol phosphate, or combinations thereof.

71. The hair protecting composition of claim 68, wherein the at least one compound having at least one phosphate group comprises phytic acid, and wherein said phytic acid is present in an amount ranging from about 0.001% to about 10%, relative to the total weight of the composition.

72. The hair protecting composition of claim 68, wherein the at least one compound having at least one phosphate group comprises glycerol phosphate, and wherein said glycerol phosphate is present in an amount ranging from about 0.001% to about 10%, relative to the total weight of the composition.

73. The hair protecting composition of claim 68, wherein the at least one compound having at least one phosphate group comprises monosodium phosphate, and wherein said monosodium phosphate is present in an amount ranging from about 0.001% to about 2%, relative to the total weight of the composition.

74. The hair protecting composition of claim 68, wherein the at least one sugar is chosen from C3-C6 monosaccharides.

75. The hair protecting composition of claim 68, wherein the at least one sugar is chosen from xylose, arabinose, ribose, 2-deoxy-ribose, allose, altrose, glucose, glucosamine, mannose, gulose, idose, galactose, talose, sorbose, psicose, fructose, tagatose, or combinations thereof.

76. The hair protecting composition of claim 68, wherein the at least one sugar is present in an amount of up to about 5%, relative to the total weight of the composition.

77. The hair protecting composition of claim 68, wherein the weight ratio of the total amount of the at least one phosphate to the total amount of the at least one sugar ranges from about 1:30 to about 30:1.

78. The hair protecting composition of claim 68, wherein the pH of the composition is less than or equal to about 7.5.

79. A method of protecting the hair or reducing heat-induced damage to hair, the method comprising applying the composition of claim 68 onto hair.

80. A method of protecting the hair or reducing heat-induced damage to hair, the method comprising:
(1) applying to the hair a hair protecting composition comprising at least one sugar, and
(2) applying to the hair a hair protecting composition comprising at least one compound having at least one phosphate group.

81. The method of claim 80, wherein the method further comprises heating the hair at a temperature of about 40° C. to about 210° C.

82. The method of claim 80, wherein the at least one compound having phosphate is present in an amount up to about 4.5%, relative to the total weight of the composition.

83. The method of claim 80, wherein the at least one compound having at least one phosphate group is chosen from monosodium phosphate, phytic acid or glycerol phosphate, or mixtures thereof.

84. The method of claim 80, wherein the at least one sugar is chosen from C3-C6 monosaccharides.

85. The method of claim 80, wherein the at least one sugar is chosen from xylose, arabinose, ribose, 2-deoxy-ribose, allose, altrose, glucose, mannose, gulose, glucosamine, idose, galactose, talose, sorbose, psicose, fructose, tagatose, or combinations thereof.

86. The method of claim 80, wherein the at least one sugar is present in an amount up to about 5%, relative to the total weight of the composition.

87. The method of claim 80, wherein the weight ratio of the total amount of the at least one phosphate to the total amount of the at least one sugar ranges from about 1:30 to about 30:1.

88. A kit comprising at least two containers, wherein a first container comprises (1) a hair protecting composition comprising at least one sugar, and a second container comprises (2) a hair protecting composition comprising at least one compound having at least one phosphate group.
